(19) United States
(12) Patent Application Publication
(10) Pub. No.: US 2009/0258940 A1
(43) Pub. Date: Oct. 15, 2009

(54) American cranberry extract and its use

(76) Inventors: Matthieu Besnard, Liffre (FR); Claude Inisan, Betton (FR); Isabelle Rousseau, Selle-en-Cogles (FR)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 12/215,533

(22) Filed: Jun. 27, 2008

(30) Foreign Application Priority Data
Apr. 9, 2008 (FR) FR 0852386

(51) Int. Cl. A61K 31/352

(52) U.S. Cl. 514/456

(57) ABSTRACT

The present invention relates to an extract derived from Vaccinium and to the process for obtaining this extract. This proanthocyanidin-rich extract can be used as a dietary or nutraceutical preparation.

### BACKGROUND OF THE INVENTION

1. Field of the Invention

The present invention relates to an extract derived from Vaccinium and to the process for obtaining this extract. This proanthocyanidin-rich extract can be used as a dietary or nutraceutical preparation.

2. Description of the Art

Vaccinium macrocarpon Aiton (American cranberry) is a small more or less creeping shrub not exceeding 30 cm and which grows spontaneously only in the east of North America from the Carolinas to Canada. It prospers on poor and marshy acid soils, among peat bogs, moors, or in conifer forests. The fruit is a small berry measuring 10 to 20 mm in diameter. At maturity, its color is bright red and its taste is slightly acid and astringent.

The American cranberry has been commonly consumed as food in North America for several hundreds of years. It does not contain any constituents in suspect quantities known for their toxicity and no incident has been reported following its consumption. At the usual doses, undesirable effects are rare. Digestive disorders constitute the main cause of harmful effects mentioned during clinical studies. They occur when the consumption of the juice becomes very excessive (3-4 liters/day) and results especially in diarrhea and gastrointestinal pain (Blumenthal M., et al., The ABC clinical guide to herbs, American Botanical Council, Austin, Tex., 2003, pp. 73-83).

The American cranberry is characterized by its richness in flavonoids and most particularly in type A proanthocyanidin oligomers, a family of compounds rarely encountered in edible plants.

Condensed tannins or proanthocyanidins are polymeric phenol compounds of flavanol units such as catechin, epicatechin and their derivatives.

The first items of information reported on the therapeutic uses of the berry are thought to date back to the 17th century. The Amerindians appear to have applied it in the form of poultices on wounds. Subsequently, navigators and colonists used it for the prevention of scurvy and for treating various symptoms: circulatory and hepatic disorders, stomach pain, fever and the like (Blumenthal et al., 2003). The use of the American cranberry in the treatment of urinary tract infections is mentioned in articles which appeared in the United States more than one hundred years ago.

It was discovered in 1984 that American cranberry juice administered orally to mice for 14 days generates urine capable of strongly inhibiting the adherence of uropathogenic strains of Escherichia coli to the uroepithelial cells (Sobota A. E., Inhibition of bacterial adherence by cranberry juice: potential use for the treatment of urinary tract infections, J. Urol., 1984, 131, 5, 1013-6).

Fractions with high concentrations of proanthocyanidins of other species of the genus Vaccinium, in particular V. myrtillus (blueberry), are thought to possess similar activities (Howell A. B., & al., Inhibition of the adherence of P-fimbriated Escherichia coli to uroepithelial-cell surfaces by proanthocyanidin extracts from cranberries, New Eng. J. Med., 1998, 339, 15, 1085; Ofek L. & al., Anti-Escherichia coli adhesion activity of cranberry and blueberry juices, New Eng. J. Med., 1991, 324, 22, 1599).

The capacities of Vaccinium macrocarpon berry extracts enriched in proanthocyanidins to inhibit the adherence of uropathogenic E. coli strains to uroepithelial cells were observed in vitro at concentrations of between 10 and 50 μg/ml (Howell et al., 1998).

During a study carried out in Canada, the tablet form containing dehydrated juice of American cranberry was considered preferable to cranberry juice because the treatment was followed to its end better, the juice having a taste that is not very well-liked by some subjects (Stothers L., A. randomised trial to evaluate effectiveness and cost effectiveness of naturopathic cranberry products as prophylaxis against urinary tract infection in women, Can. J. Urol., 2002, 9, 3, 1558-62).

However, the low proanthocyanidin dosage of these tablets forced patients to ingest several tablets per day, making this treatment tedious.

In its 2003-SA-0352 opinion of Apr. 6, 2004, Agence Française de Sécurité Sanitaire des Aliments (AFSSA) (French Agency for Food Safety) approved the following claim: “Le jus concentré ou la poudre de jus de fruits de Vaccinium macrocarpon contribue à diminuer la fixation de certaines bactéries Escherichia coli sur les parois des voies urinaires.” (The concentrated juice or the juice powder of Vaccinium macrocarpon fruits contributes toward reducing the attachment of certain Escherichia coli bacteria to the walls of the urinary tracts). AFSSA thus considers that the claim “contribue à diminuer la fixation de certaines bactéries E. coli sur les parois des voies urinaires” (contributes toward reducing the attachment of certain E. coli bacteria to the walls of the urinary tracts) can be accepted for fresh/frozen cranberry and cranberry purée provided that there is a daily consumption of quantities of the product providing at least 36 mg of proanthocyanidins (AFSSA, 2007).

Accordingly, a need remains for compositions based on cranberry, rich in proanthocyanidins, that are easy to consume by a human being in a daily dose without the constraints of the treatment in terms of taste of the product to be ingested or of number of tablets to be taken weighing too heavily on this patient, making it easy to follow said treatment daily.

### SUMMARY OF THE INVENTION

The applicant has developed novel Vaccinium extracts which constitute the subject of the invention.

The subject of the invention is also the process for obtaining this fraction.

Another subject consists in the application of this extract as a dietary or nutraceutical supplement.

Other subjects will emerge on reading the description and the examples which follow.

### DESCRIPTION OF THE FIGURE

FIG. 1 shows the quantity of bacteria attached to the plate expressed as optical density as a function of the cranberry extract tested (0=without extract, batch 9030=AFCANN 9030 extract from Diana Naturals, batch A4124A=extract available on market) and of the bacterium used (DH5α strain=reference strain, strains 5 and 6=uropathogenic strains).

### DETAILED DESCRIPTION OF THE INVENTION

The extract in accordance with the invention is derived from plants of the genus Vaccinium, comprising at least 15% by weight of proanthocyanidins expressed as procyanidin C1 equivalent relative to the dry weight of the extract.

Preferably, the vaccinium is Vaccinium macrocarpon or American cranberry. This extract is preferably obtained from the berry of this plant.

Preferably, this extract contains more than 5% sugar obtained from the fruit relative to the dry weight of the extract.

Preferably, this extract has a mean degree of polymerization of the proanthocyanidins (that is to say a mean length of the tannin chain) greater than 5. More preferably, this degree is greater than 6. More preferably still, the mean degree of polymerization of the proanthocyanidins in the extract of the invention is greater than 8.

Preferably, this cranberry extract preserves a quantity of anthocyanins identical to that of cranberry juice.

Since the process for producing the proanthocyanidin-rich extract according to the invention does not modify the proportions of anthocyanins of the cranberry, this characteristic of the cranberry extract therefore remains close to those of the juice before treatment.

The process for producing an extract according to the invention is characterized in that the American cranberry fruit is placed in an aqueous solution, in that a solid-liquid separation is performed, in that an organic solvent is added and then in that the product obtained is dehydrated in order to obtain the extract according to the invention.

Preferably, the pH of the aqueous solution is between 2 and 6, the temperature is less than 95° C. and the pressure is between 1 and 3 atmospheres.

Optionally, sulfite or disulfite is added during the aqueous extraction phase at concentrations of between 0.01 and 1% of the volume of the aqueous solution, and preferably at a concentration of 0.3%. Sodium sulfite, calcium sulfite, potassium sulfite, sodium disulfite or potassium disulfite may be used.

By its antioxidant and antiseptic action, the sulfite or disulfite contributes to preserving the activity of the extract according to the invention during the production process.

Preferably, the solid-liquid separation is performed at a pH of between 2 and 7, at a temperature of between 15 and 70° C. and at a pressure of between 1 and 15 atmospheres.

Preferably, the organic solvent is added in proportions of 0 to 15 by volume relative to the volume of the cranberry in aqueous solution.

Preferably, the organic solvent used is an alcohol, an aldehyde or an ester.

More preferably, the organic solvent added is ethanol, acetaldehyde or ethyl acetate.

The product obtained is preferably dehydrated under reduced pressure, that is to say below 1 atmosphere, at a temperature of less than 60° C.

Optionally, a final dehydration step is performed at a temperature of between 80 and 190° C. and a pressure of between 0.5 and 2 atmospheres.

The extract of the invention is obtained by the process described above.

This American cranberry extract having the stated characteristics, liable to be obtained according to the process described above, can be used as a dietary or nutraceutical supplement.

The proanthocyanidin-rich extract according to the invention has the characteristic feature of preventing the adherence of bacteria, and more particularly of E. coli bacteria, to the surface of the urinary tract walls.

The subject of the invention is also a composition comprising, inter alia, the proanthocyanidin-rich extract described above.

The compositions according to the invention can be ingested. According to the mode of administration, the composition according to the invention may be provided in all the forms normally used in the food of nutraceutical sector.

The compositions according to the invention may in particular be provided in the form of gelatin capsules, tablets, powders or drinks.

The nutraceutical or dietary compositions of the present invention are conventionally formulated according to the applications for which they are intended.

### EXAMPLES

The following examples illustrate the invention without limiting it in any manner.

### Example 1

### Process for Producing the AFCANN9030 Extract According to the Invention

American cranberries were placed in solution in an aqueous phase in an amount of one volume of berries per 5 volumes of water. After grinding and solid-liquid separation by decantation at a temperature of 25° C., ethyl acetate is added in an amount of 5 volumes of acetate per one volume of cranberry in an aqueous phase, at a temperature of 20° C., a pH of 4.5, under a pressure of 1 atm, and then the two phases obtained are separated. Dehydration is then performed under a reduced pressure of 0.1 to 0.5 atm, at a temperature of 30 to 40° C., and then a second dehydration at a pressure of 0.9 to 1.5 atm and a temperature of 145° C.

The AFCANN9030 product obtained is a purple powder.

### Example 2

### Assay of Proanthocyanidins in the AFCANN9030 Extract Obtained

The Bate-Smith method modified by Porter for the assay of proanthocyanidins was used (Bate-Smith E. C. Phytochemistry, 1973, 12, pp 907-912 and Porter. L. J., & al., Phytochemistry, 1986, 25, 223-230).

The assay reaction is performed in two stages. In a first instance, the flavan-3-ol polymers are hydrolyzed in a butanol-HCl medium. The hydrolysis is complete and releases monomers of the catechin and epicatechin type. The second assay step consists in oxidizing these monomers under the action of Fe^{III}. The oxidation product is cyanidin and is quantified using a spectrophotometer at 541 nm.

The Bate-Smith reagent is prepared by mixing 500 mL of HCl at the concentration of 35%, 500 mL of n-butanol and 150 mg of Fe₂(SO₄)₃.

100 mg of American cranberry extract are dissolved in 100 mL of the mixture of demineralized water and ethanol 1/1 by weight. 2 mL of this solution are mixed with 6 mL of Bate-Smith reagent in a first tube. Half of the solution is transferred into a second tube. The first tube is left in the dark while the second tube is heated for 30 minutes on a water bath at the temperature of 100° C. and then left in the dark for 10 minutes. The measurement of the absorbance values (OD) of the two tubes is performed at 541 nm by making a blank with deionized water.

The results are expressed as proanthocyanidin C1 equivalent according to the following formula.

$$\% proanthocyanidins = \frac{( [ OD 541 tube 2 - OD 541 tube 1 ] - 0.0183 ) \times 100}{5.1407 \times concentration of extract in g \text{/} L}$$

The measurement of the proanthocyanidin concentration of the AFCANN9030 extract is 30%±2 expressed as proanthocyanidin C1 equivalent.

It should be noted that the quantification of the proanthocyanidins takes into account the anthocyanins initially present in the medium and the absorption due to these anthocyanins is subtracted from the absorption read after the Porter reaction.

### Example 3

### Characterization of the Extract of Example 1 (AFCANN9030)

| | Extract of example 1 |
|---|---|
| % of proanthocyanidins eq procyanidin C1 | 30.5% |
| % TPP eq catechin (UV 280 nm) | 17.3% |
| % flavanols eq rutin (UV 354 nm) | 2.4% |
| % flavanols eq quercetin (UV 354 nm) | 1.4% |
| Fructose (batch 3808001) | 24.4 mg/g |
| Glucose (batch 3808001) | 110.8 mg/g |
| Sucrose (batch 3808001) | ND |
| Total dry residue | 97.5% |
| Protein (N * 6.25) Dumas | 2.7% |
| Fatty substances by hydrolysis | <1% |
| Inorganic matter | 2.4% |
| Total dietary fiber | 13.5% |

eq: equivalent
TPP: total polyphenols

### Example 4

### Composition of the Extract Obtained in Example 1 (AFCANN9030)

Thioacidolysis is another technique which makes it possible to determine the proanthocyanidin content and the mean degree of polymerization of the proanthocyanidins (Guyot et al., J. Agric. Food Chem., 1998, 1698-1705).

This technique is also based on the hydrolysis of the proanthocyanidolic polymers in an acid medium (HCl). The α-toluenethiol present in the reaction medium reacts with the monomers released during the hydrolysis and the compounds formed are quantified by liquid chromatography (HPLC).

Results of the analyses after thioacidolysis

| | Extract of example 1 |
|---|---|
| mDP | 8.5 |
| Tannin content | 191.5 |
| mg/g powder | |
| % epi | 71.5 |
| % cat | 0.9 |
| % dimA | 27.6 |

mDP mean degree of polymerization (mean length of the tannin chain):
% epi = % epicatechin
% cat = % catechin
% dimA = % dimer of type A

### Example 5

### Test of Adhesion of the Extract According to the Invention to a Biofilm

The DH5α bacterial strain which does not possess fimbriae is used in this test as negative control for adhesion. The bacterial strains 5 and 6 are uropathogenic E. coli strains obtained from clinical isolates.

The strains are cultured in CFA medium at 37° C. The composition of this culture medium is the following: 10 grams of casamino acids (Difco), 1.5 g of yeast extract, 0.05 g of MgSO₄ and 0.005 g of MnCl₂ are added to 1 liter of deionized water.

For each bacterial strain, the adhesion of the strain alone, or in the presence either of cranberry from batch AFCANN9030 according to the invention (Diana Naturals), containing 30% of proanthocyanidins, or of cranberry from batch A4124A (commercial sample), containing 3% of proanthocyanidins, is tested. The cranberry samples are prepared so as to obtain a proanthocyanidin concentration of 500 μg/mL, that is a dilution of 16.7 mg of batch 9030 or of 167 mg of batch A4124A in 10 mL of water. These solutions are then diluted 10-fold in water in order to carry out the tests.

An overnight culture of the bacterium to be studied is used, and 0.2 mL of this culture is deposited in 96-well microplate (Greiner) with or without the cranberry sample to be tested, again diluted 5-fold in water. Each test is performed in triplicate. As negative control for the culture, CFA medium not inoculated with either of their cranberry samples to be tested is deposited.

This microplate is placed in an incubator at 35° C. for 24 h.

The following day, after removing the culture medium and washing the wells with distilled water, 200 μL of methanol are added to each well for 15 min. After removing the methanol, the plate is dried in an incubator for 30 min. Next, 200 μL of crystal violet (Sigma) are added for 10 min. After removing the stain and washing the wells with distilled water, 200 μL of acetic acid/ethanol solution (vol/vol) are added in order to destain the biofilm produced. The reading is performed with a spectrophotometer at a wavelength of 570 nm. The higher the value of the optical density, the higher the number of bacteria attached to the plate.

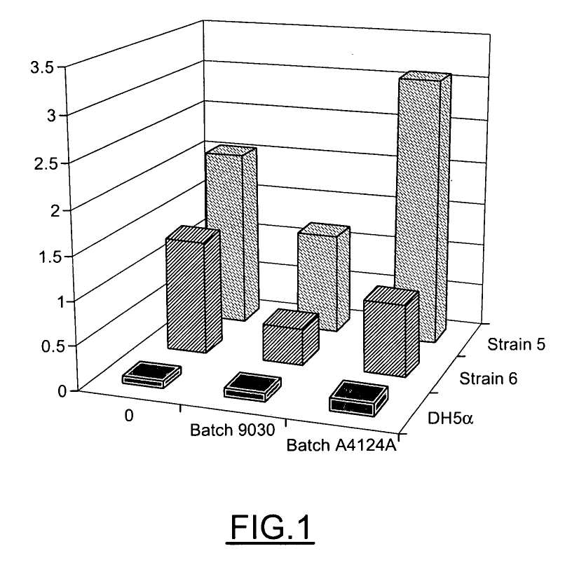

The results of FIG. 1 show that the extract according to the invention tested has an optical density less than that of the extract A4124A, therefore an adhesion of bacteria to the plate well less than that of batch A4124A regardless of the bacterium tested. Now, the proanthocyanidin concentration in the culture medium is the same in the test with the extract according to the invention and the test with the A4124A extract. The effect of inhibition of adhesion of the bacteria to the microplate attributed to the proanthocyanidins present in the medium at equivalent concentrations is therefore higher for the extract according to the invention than for the A4124A extract, even when the proanthocyanidin concentrations are equivalent. Thus, the proanthocyanidins present in the extract according to the invention have a higher effect on the inhibition of the adhesion of bacteria than the A4124A extract at the same concentration by mass.

It is known that this commercial extract (batch A4124A) has a mean degree of polymerization of proanthocyanidins (that is to say a mean length of tannin chain) of the order of 3 to 4. Moreover, the extract according to the invention has a mean degree of polymerization of proanthocyanidins of 8.1.

It is therefore possible to deduce from these results that the mean degree of polymerization of proanthocyanidins in the cranberry extract according to the invention plays a role in the adhesion of uropathogenic bacteria to the surface of the well. Thus, the extract according to the invention has an increased antiadhesive efficacy compared with the commercial product A4124A.

### Example 6

### Example of Formulation of a Gelatin Capsule

For the manufacture of one gelatin capsule of 250 mg in total, the following are mixed:

240 mg of extract according to the invention with a titer of 15% proanthocyanidins expressed as procyanidin C1 equivalent relative to the dry weight of the extract,

10 mg of microcrystalline cellulose.

Each gelatin capsule contains 36 mg of proanthocyanidins.

### Example 7

### Example of Formulation of a Gelatin Capsule

For the manufacture of one gelatin capsule of 300 mg in total, the following are mixed:

200 mg of AFCANN9030 extract (with a titer of 30.5% proanthocyanidins expressed as procyanidin C1 equivalent relative to the dry weight of the extract),

100 mg of microcrystalline cellulose.

Each gelatin capsule contains 61 mg of proanthocyanidins.

### Example 8

### Example of a Drink According to the Invention

100 grams of the following composition are prepared:

| | Quantity in grams |
|---|---|
| AFCANN9030 extract | 0.68 |
| Powder of clear raspberry juice(sold under the name | 56.3 |
| AFFRAM0013 from Diana Naturals) | |
| Powder of clear strawberry juice (sold under the name | 29.3 |
| AFFRAI0013 from Diana Naturals) | |
| Powder of blackcurrant juice (sold under the name | 11.3 |
| AFCASS9003 from Diana Naturals) | |
| Sucralose | 0.23 |
| Citric acid | 2.25 |

To prepare the drink, 22 grams of this powdered composition are then mixed in a final volume of 500 ml of water. After stirring, a drink with a pronounced raspberry taste is ready to be consumed.

A daily ration of 500 mL of this drink provides 45 milligrams of proanthocyanidins.

What is claimed is:

1. An extract derived from plants of the genus Vaccinium, which comprises at least 15% by weight of proanthocyanidins expressed as procyanidin C1 equivalent relative to the dry weight of the extract.

2. The extract as claimed in claim 1, in which the plant of the genus Vaccinium is Vaccinium macrocarpon or American cranberry.

3. The extract as claimed in claim 1, wherein the berry of American cranberry is used.

4. The extract as claimed in claim 1, wherein the mean degree of polymerization of the proanthocyanidins is greater than 5.

5. The extract as claimed in claim 4, wherein the mean degree of polymerization of the proanthocyanidins is greater than 6.

6. The extract as claimed in claim 5, wherein the mean degree of polymerization of the proanthocyanidins is greater than 8.

7. A process for preparing an extract as claimed in claim 1, wherein the American cranberry is placed in an aqueous solution, a solid-liquid separation is performed, an organic solvent is added and then the product obtained is dehydrated in order to obtain the extract.

8. The process for preparing an extract as claimed in claim 7, in which a second dehydration is performed.

9. The process for preparing an extract as claimed in claims 7, wherein the organic solvent is an alcohol.

10. The process for preparing an extract as claimed in claim 9, in which the alcohol is ethanol.

11. The process for preparing an extract as claimed in claim 7, wherein the organic solvent is an aldehyde.

12. The process for preparing an extract as claimed in claim 11, in which the aldehyde is acetaldehyde.

13. The process for preparing an extract as claimed in claim 7, wherein the organic solvent is an ester.

14. The process for preparing an extract as claimed in claim 13, in which the ester is ethyl acetate.

15. The process as claimed in claim 7, wherein the solvent is added in proportions of 0 to 15 by volume relative to the volume of cranberry in aqueous solution.

16. The extract as claimed in claim 1, which is obtained by the process as defined in claim 7.

17. A dietary or nutraceutical composition, which contains an extract as defined in claim 1.
